(19) United States
(12) Patent Application Publication
(10) Pub. No.: US 2010/0047234 A1
(43) Pub. Date: Feb. 25, 2010

(54) TREATING SKIN CANCER

(76) Inventor: Svetomir N. Markovic, Rochester, MN (US)

Correspondence Address: FISH & RICHARDSON P.C., PO BOX 1022, MINNEAPOLIS, MN, 55440-1022, US

(21) Appl. No.: 12/295,834

(22) PCT Filed: Mar. 14, 2008
(86) PCT No.: PCT/US2008/057025

§ 371 Date: Sep. 17, 2009

Related U.S. Application Data
(60) Provisional application No. 60/894,780, filed on Mar. 14, 2007.

(51) Int. Cl. A61K 39/395

(52) U.S. Cl. 424/133.1

(57) ABSTRACT

This document provides methods and materials related to treating skin cancer. For example, methods and materials relating to the use of a taxane compound, an alkylating agent, and an anti-VEGF polypeptide antibody to treat skin cancer are provided.

### CROSS-REFERENCE TO RELATED APPLICATIONS

This application claims the benefit of U.S. Provisional Patent Application No. 60/894,780, filed on Mar. 14, 2007, which is incorporated by reference in its entirety herein.

### STATEMENT AS TO FEDERALLY SPONSORED RESEARCH

This invention was made with government support under CA025224 awarded by National Institutes of Health. The government has certain rights in the invention.

### BACKGROUND

1. Technical Field

This document relates to methods and materials involved in treating skin cancer (e.g., melanoma). For example, this document relates to methods and materials involved in using a taxane compound (e.g., paclitaxel), an alkylating agent (e.g., carboplatin), and an anti-VEGF polypeptide antibody to treat skin cancer.

2. Background Information

Melanoma is the most serious form of skin cancer. It is a malignant tumor that originates in melanocytes, the cells which produce the pigment melanin that colors skin, hair, and eyes and is heavily concentrated in most moles. While it is not the most common type of skin cancer, melanoma underlies the majority of skin cancer-related deaths. About 48,000 deaths worldwide are registered annually as being due to malignant melanoma. Worldwide, there are about 160,000 new cases of melanoma each year. Melanoma is more frequent in white men and is particularly common in white populations living in sunny climates. Other risk factors for developing melanoma include a history of sunburn, excessive sun exposure, living in a sunny climate or at high altitude, having many moles or large moles, and a family or personal history of skin cancer.

Melanomas fall into four major categories. Superficial spreading melanoma can travel along the top layer of the skin before penetrating more deeply. Lentigo maligna typically appears as a flat or mildly elevated mottled tan, brown, or dark brown discoloration and is found most often in the elderly. Nodular melanoma can occur anywhere on the body as a dark, protuberant papule or a plaque that varies from pearl to gray to black. Acral-lentiginous melanoma, although uncommon, is the most common form of melanoma in blacks. It can arise on palmar, plantar, or subungual skin. Metastasis of melanoma occurs via lymphatics and blood vessels. Local metastasis results in the formation of nearby satellite papules or nodules that may or may not be pigmented. Direct metastasis to skin or internal organs can occur.

### SUMMARY

This document provides methods and materials related to treating skin cancer. For example, this document provides methods and materials for using a taxane compound (e.g., paclitaxel), an alkylating agent (e.g., carboplatin), and an anti-VEGF polypeptide antibody to treat skin cancer (e.g., melanoma). The methods and materials provided herein can be used to increase the progression-free survival rate, or to increase the time to progression, in skin cancer patients. Increasing progression-free survival or time to progression can allow skin cancer patients to live longer.

In general, one aspect of this document features a method for treating a mammal having skin cancer. The method comprises, or consists essentially of, administering to the mammal a taxane compound, an alkylating agent, and an anti-VEGF polypeptide antibody under conditions where the length of progression-free survival is increased. The mammal can be a human. The skin cancer can be melanoma. The skin cancer can be stage IV melanoma. A composition comprising the taxane compound, the alkylating agent, and the anti-VEGF polypeptide antibody can be administered to the mammal. The anti-VEGF polypeptide antibody can be administered first. The taxane compound can be administered after the anti-VEGF polypeptide antibody. The alkylating agent can be administered after the taxane compound. The taxane compound can be paclitaxel. The alkylating agent can be a platinum compound, such as carboplatin. The anti-VEGF polypeptide antibody can be a humanized antibody. The anti-VEGF polypeptide antibody can be bevacizumab. The taxane compound, the alkylating agent, and the anti-VEGF polypeptide antibody can be administered by injection. The taxane compound can be administered more frequently than the anti-VEGF polypeptide antibody, and the anti-VEGF polypeptide antibody can be administered more frequently than the alkylating agent. Each of the taxane compound, the alkylating agent, and the anti-VEGF polypeptide antibody can be administered within a 60 day time period. The progression-free survival can be increased by 25 percent. The progression-free survival can be increased by 50 percent. The progression-free survival can be increased by 75 percent. The progression-free survival can be increased by 100 percent. The taxane compound, the alkylating agent, and the anti-VEGF polypeptide antibody can be administered under conditions where the time to progression is increased.

In another aspect, this document features a composition. The composition comprises, or consists essentially of, a taxane compound, an alkylating agent, and an anti-VEGF polypeptide antibody.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DETAILED DESCRIPTION

This document provides methods and materials related to treating skin cancer in mammals. For example, this document provides methods and materials related to the use of a taxane compound (e.g., paclitaxel), an alkylating agent (e.g., carboplatin), and an anti-VEGF polypeptide antibody to treat skin cancer in a mammal. The methods and materials provided herein can be used to treat skin cancer in any type of mammal including, without limitation, mice, rats, dogs, cats, horses, cows, pigs, monkeys, and humans. Any type of skin cancer, such as melanoma, can be treated. For example, stage I, stage II, stage III, or stage IV melanoma can be treated. In some cases, a lymph node positive, a lymph node negative, or a metastatic melanoma can be treated.

In general, skin cancer can be treated by administering a taxane compound, an alkylating agent, and an anti-VEGF polypeptide antibody to a mammal having skin cancer. It will be appreciated that a taxane compound, an alkylating agent, and an anti-VEGF polypeptide antibody can be used to treat skin cancer upon administration either individually or in combination. For example, a mammal having skin cancer can be treated by administering a taxane compound, an alkylating agent, and an anti-VEGF polypeptide antibody individually. In some embodiments, an anti-VEGF polypeptide antibody can be administered first. In some embodiments, a taxane compound is administered after an anti-VEGF polypeptide antibody. In some embodiments, an alkylating agent can be administered after a taxane compound. In some cases, skin cancer can be treated by administering a composition containing a taxane compound, an alkylating agent, and an anti-VEGF polypeptide antibody to a mammal.

Any taxane compound, such as Paclitaxel (Taxol®) or docetaxol (Taxotere®), can be used to treat skin cancer. Any alkylating agent also can be used to treat skin cancer. For example, a platinum compound, such as Carboplatin (Paraplatin®), cisplatin (Platinol®), oxaliplatin (Eloxatin®), or BBR3464 can be used. In addition, any anti-VEGF polypeptide antibody, such as bevacizumab (Avastin®), can be used.

Any appropriate method can be used to administer a taxane compound, an alkylating agent, and an anti-VEGF polypeptide antibody to a mammal. For example, a taxane compound, an alkylating agent, and an anti-VEGF polypeptide antibody can be administered orally or via injection (e.g., subcutaneous injection, intramuscular injection, intravenous injection, or intrathecal injection). In some cases, a taxane compound, an alkylating agent, and an anti-VEGF polypeptide antibody can be administered by different routes. For example, a taxane compound and an alkylating agent can be administered orally and an anti-VEGF polypeptide antibody can be administered via injection.

Before administering a taxane compound, an alkylating agent, and an anti-VEGF polypeptide antibody to a mammal, the mammal can be assessed to determine whether or not the mammal has skin cancer. Any appropriate method can be used to determine whether or not a mammal has skin cancer. For example, a mammal (e.g., human) can be identified as having skin cancer using standard diagnostic techniques. In some cases, a tissue biopsy can be collected and analyzed to determine whether or not a mammal has skin cancer.

After identifying a mammal as having skin cancer, the mammal can be administered a taxane compound, an alkylating agent, and an anti-VEGF polypeptide antibody. For example, a taxane compound, an alkylating agent, and an anti-VEGF polypeptide antibody can be administered prior to or in lieu of surgical resection of a tumor. In some cases, a taxane compound, an alkylating agent, and an anti-VEGF polypeptide antibody can be administered following resection of a tumor. A taxane compound, an alkylating agent, and an anti-VEGF polypeptide antibody can be administered to a mammal in any amount, at any frequency, and for any duration effective to achieve a desired outcome (e.g., to increase progression-free survival or to increase the time to progression). In some cases, a taxane compound, an alkylating agent, and an anti-VEGF polypeptide antibody can be administered to a mammal having skin cancer to reduce the progression rate of melanoma by 5, 10, 25, 50, 75, 100, or more percent. For example, the progression rate can be reduced such that no additional cancer progression is detected. Any method can be used to determine whether or not the progression rate of skin cancer is reduced. For example, the progression rate of skin cancer can be assessed by imaging tissue at different time points and determining the amount of cancer cells present. The amounts of cancer cells determined within tissue at different times can be compared to determine the progression rate. After treatment as described herein, the progression rate can be determined again over another time interval. In some cases, the stage of skin cancer after treatment can be determined and compared to the stage before treatment to determine whether or not the progression rate was reduced.

In some cases, a taxane compound, an alkylating agent, and an anti-VEGF polypeptide antibody can be administered to a mammal having skin cancer under conditions where progression-free survival or time to progression is increased (e.g., by 5, 10, 25, 50, 75, 100, or more percent) as compared to the median progression-free survival or time to progression, respectively, of corresponding mammals having untreated skin cancer. In some cases, a taxane compound, an alkylating agent, and an anti-VEGF polypeptide antibody can be administered to a mammal having skin cancer to increase progression-free survival or time to progression by 5, 10, 25, 50, 75, 100, or more percent as compared to the median progression-free survival or time to progression, respectively, of corresponding mammals having skin cancer and having received a taxane compound and an alkylating agent alone. Progression-free survival and time to progression can be increased by any amount (e.g., 5%, 7.5%, 10%, 25%, 50%, 75%, 100%, or more). In addition, progression-free survival can be measured over any length of time (e.g., one month, two months, three months, four months, five months, six months or longer).

In some cases, a taxane compound, an alkylating agent, and an anti-VEGF polypeptide antibody can be administered to a mammal having skin cancer under conditions where the 8-week progression-free survival rate for a population of mammals is 65% or greater (e.g., 66%, 67%, 68%, 69%, 70%, 71%, 72%, 73%, 74%, 75%, 76%, 77%, 78%, 79%, 80% or greater) than that observed in a population of comparable mammals not receiving a taxane compound, an alkylating agent, and an anti-VEGF polypeptide antibody. In some cases, a taxane compound, an alkylating agent, and an anti-VEGF polypeptide antibody can be administered to a mammal having skin cancer under conditions where the median time to progression for a population of mammals is at least 150 days (e.g., at least 155, 160, 163, 165, or 170 days).

An effective amount of a taxane compound, an alkylating agent, and an anti-VEGF polypeptide antibody can be any amount that reduces the progression rate of skin cancer, increases the progression-free survival rate, or increases the median time to progression without producing significant toxicity to the mammal. Typically, an effective amount of a taxane compound such as paclitaxel can be from about 50 mg/m² to about 150 mg/m² (e.g., about 80 mg/m²), an effective amount of an alkylating agent such as carboplatin can be from about AUC 1 to about AUC 5 or from about AUC 1 to about AUC 10 (e.g., about AUC 6, about AUC 5, about AUC 4, about AUC 3, about AUC 2, or about AUC 1), and an effective amount of an anti-VEGF polypeptide antibody such as bevacizumab can be from about 5 mg/kg to about 20 mg/kg (e.g., about 10 mg/kg). If a particular mammal fails to respond to a particular amount, then the amount of one or more of the taxane compound, alkylating agent, and anti-VEGF polypeptide antibody can be increased by, for example, two fold. After receiving this higher concentration, the mammal can be monitored for both responsiveness to the treatment and toxicity symptoms, and adjustments made accordingly. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the skin cancer may require an increase or decrease in the actual effective amount administered.

The frequency of administration can be any frequency that reduces the progression rate of skin cancer, increases the progression-free survival rate, or increases the median time to progression without producing significant toxicity to the mammal. For example, the frequency of administration can be from about once a month to about three times a month, or from about twice a month to about six times a month, or from about once every two months to about three times every two months. The frequency of administration can remain constant or can be variable during the duration of treatment. In addition, the frequency of administration of a taxane compound, an alkylating agent, and an anti-VEGF polypeptide antibody can be the same or can differ. For example, a taxane compound can be administered three times during a 28 day period, while an alkylating agent can be administered one time and an anti-VEGF polypeptide antibody can be administered two times during the same period. In some cases, a taxane compound can be administered on day 1, 8, and 15 of a 28 day cycle, an alkylating agent can be administered on day 1 of the 28 day cycle, and an anti-VEGF polypeptide antibody can be administered on day 1 and 15 of the 28 day cycle. A course of treatment with a taxane compound, an alkylating agent, and an anti-VEGF polypeptide antibody can include rest periods. For example, a taxane compound, an alkylating agent, and an anti-VEGF polypeptide antibody can be administered over a two week period followed by a two week rest period, and such a regimen can be repeated multiple times. As with the effective amount, various factors can influence the actual frequency of administration used for a particular application. For example, the effective amount, duration of treatment, use of multiple treatment agents, route of administration, and severity of the skin cancer may require an increase or decrease in administration frequency.

An effective duration for administering a composition provided herein can be any duration that reduces the progression rate of skin cancer, increases the progression-free survival rate, or increases the median time to progression without producing significant toxicity to the mammal. Thus, the effective duration can vary from several days to several weeks, months, or years. In general, the effective duration for the treatment of skin cancer can range in duration from several weeks to several months. In some cases, an effective duration can be for as long as an individual mammal is alive. Multiple factors can influence the actual effective duration used for a particular treatment. For example, an effective duration can vary with the frequency of administration, effective amount, use of multiple treatment agents, route of administration, and severity of the skin cancer.

A composition containing a taxane compound, an alkylating agent, and an anti-VEGF polypeptide antibody can be in any appropriate form. For example, a composition provided herein can be in the form of a solution or powder with or without a diluent to make an injectable suspension. A composition also can contain additional ingredients including, without limitation, pharmaceutically acceptable vehicles. A pharmaceutically acceptable vehicle can be, for example, saline, water, lactic acid, and mannitol.

After administering a composition provided herein to a mammal, the mammal can be monitored to determine whether or not the skin cancer was treated. For example, a mammal can be assessed after treatment to determine whether or not the progression rate of melanoma was reduced (e.g., stopped). As described herein, any method can be used to assess progression and survival rates.

The invention will be further described in the following examples, which do not limit the scope of the invention described in the claims.

### EXAMPLES

### Example 1

### Treating Unresectable Stage IV Melanoma with Carboplatin, Paclitaxel and Bevacizumab

A two-stage clinical trial was conducted in patients with unresectable stage IV melanoma to assess the anti-tumor activity and toxicity profile of the combination of paclitaxel (80 mg/m² IV on days 1, 8, and 15 of a 28-day cycle), carboplatin (AUC=6 IV on day 1), and bevacizumab (10 mg/kg IV on days 1 and 15). Treatment was continued until progression or intolerable toxicity. Eligible patients had measurable disease by RECIST (Response Evaluation Criteria in Solid Tumors), were at least 18 years of age with good performance status (0-2), life expectancy of at least 4 months, and had acceptable pre-registration blood and urine tests. Exclusion criteria included: cancer therapy less than four weeks prior to registration; prior chemotherapy with carboplatin, paclitaxel, or agents know to disrupt VEGF activity; more than one prior systemic chemotherapy; failure to recover from prior therapy; known central nervous system metastases; tumor invasion of major blood vessels; active infection requiring parenteral antibiotics; significant recent bleeding; major surgical procedures; uncontrolled hypertension; and ongoing anticoagulation. Pregnant or nursing women were not eligible.

Patients were treated with bevacizumab 10 mg/kg IV on days 1 and 15 of a 28-day cycle, paclitaxel 80 mg/m² IV on days 1, 8 and 15, and carboplatin AUC 6 IV on day 1. Paclitaxel was administered over 1 hour after completion of bevacizumab infusion. Carboplatin was administered over 15 to 30 minutes after completion of the paclitaxel infusion.

Within 14 days of registration, patients underwent a complete physical exam, assessment of ECOG PS, complete blood cell count (CBC), comprehensive metabolic panel including lactic dehydrogenase (LDH), measurement of UPC ratio and baseline tumor assessment (by CT scan, MRI, etc). All patients underwent a brain MRI to rule out brain metastases. Blood was also drawn to measure baseline VEGF levels and several markers of immune homeostasis. VEGF tissue expression was determined in patients with available archival primary or metastatic tumor specimens. After initiation of treatment, physical exams, toxicity evaluations and serum chemistries were repeated every 4 weeks. CBC was measured weekly during treatment. UPC ratio was measured ≦48 hours prior to each bevacizumab infusion. Additional blood was drawn for correlative studies (VEGF levels and markers of immune homeostasis) on day 1 of cycles 2, 3, 4 and every even cycle thereafter during treatment and at discontinuation of treatment. Tumor status was assessed every 8 weeks during treatment using RECIST criteria.

The primary endpoint of this trial was the 8-week progression-free survival (PFS) rate, which is defined as the number of eligible patients who remain progression-free and on study treatment for at least 56 days post-registration divided by the number of eligible patients who began treatment. If a patient died without documentation of disease progression, that patient was considered to have progressed at death unless there was sufficient evidence to the contrary.

Sixty percent of the patients were male. The patients had a median age of 55 (30-84) and a good performance status (85% had PS of 0). Prior therapies included immunotherapy (52.8%), radiation therapy (26.4%), chemotherapy (24.5%) or vaccines (13.2%). Twenty-three percent of patients had stage M1a, fifteen percent had stage M1b, and sixty-two percent had stage M1c. See Table 1 for patient characteristics.

TABLE 1
Selected Demographic and Baseline Characteristics

| Patient Characteristics | n = 53 |
|---|---|
| Median Age (range) | 55 (range 30-84) |
| Male | 62.3% |
| M Stage | |
| M1a | 23% |
| M1b | 15% |
| M1c | 63% |
| Prior Systemic Therapies | |
| None | 28.3% |
| Immunotherapy | 52.8% |
| Radiation therapy | 26.4% |
| Chemotherapy | 24.5% |
| Vaccine therapy | 13.2% |
| ECOG Performance Status | |
| 0 | 81.1% |
| 1 | 17.0% |
| 2 | 1.9% |
| Pre-existing Signs and Symptoms | |
| Grade 3 hypertension | 1.9% |
| Grade 2 fatigue | 7.6% |
| Grade 2 anorexia | 5.6% |
| Grade 2 nausea | 1.9% |
| Grade 2 musculoskeletal pain | 1.9% |

The most common severe (≧grade 3) hematologic toxicities were neutropenia (53%), leukopenia (38%), thrombocytopenia (11%) and anemia (8%). The most common severe non-hematologic toxicities were hypertension (9%), nausea (6%), and fatigue (6%) (Table 2). Five patients discontinued study treatment due to adverse events including reaction to carboplatin (1 patient), sensory neuropathy (1 patient), motor neuropathy (1 patient), hypertension and proteinuria (1 patient) and catheter-related infection (1 patient). A 62 year-old female died while on study. She had achieved a partial response after 8 cycles of treatment when she presented to the emergency room with neurologic symptoms. Brain imaging revealed that she had bled into previously undiagnosed brain metastases. She died 3 days later.

TABLE 2
Most common severe toxicities reported

| | Any | |
|---|---|---|
| Toxicity | Grade | ≧grade 3 |
| Neutropenia | 75% | 53% |
| Leukopenia | 79% | 38% |
| Anemia | 92% | 8% |
| Thrombocytopenia | 64% | 11% |
| Fatigue | 89% | 6% |
| Nausea | 53% | 6% |
| Hypertension | 30% | 9% |
| Infection including | 17% | 8% |
| bladder catheter-related, febrile neutropenia, nail | | |
| bed, pharynx, sinus, upper airway, urinary tract, | | |
| NOS | | |
| Hemorrhage including epistaxis, anal, broncho- | 62% | 2% |
| pulmonary, CNS, intra-abdominal, nasal, rectal, | | |
| urogenial, vaginal, NOS | | |

Patients were followed for a minimum of 7.5 months or until death. Among the 53 patients enrolled, 8 (15%) achieved a partial remission (PR) and an additional 31 (58%) completed 2 cycles of treatment with stable disease. No complete remissions (CR) were observed. At last follow-up, 3 patients continue on study treatment, 7 are alive off study treatment without progression, 10 are alive with disease progression, and 31 have died of disease. The estimated 8-week progression-free survival (PFS) rate was 85% (95% CI: 76-95%) with a median PFS of 181 days (6 months). Median overall survival (OS) was 11.4 months.

The results indicated that the combination of paclitaxel, carboplatin, and bevacizumab was well tolerated and clinically active in patients with stage IV melanoma.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating a mammal having skin cancer, said method comprising administering to said mammal a taxane compound, an alkylating agent, and an anti-VEGF polypeptide antibody under conditions wherein the length of progression-free survival is increased.

2. The method of claim 1, wherein said mammal is a human.

3. The method of claim 1, wherein said skin cancer is melanoma.

4. The method of claim 1, wherein said skin cancer is stage IV melanoma.

5. The method of claim 1, wherein a composition comprising said taxane compound, said alkylating agent, and said anti-VEGF polypeptide antibody is administered to said mammal.

6. The method of claim 1, wherein the anti-VEGF polypeptide antibody is administered first.

7. The method of claim 1, wherein the taxane compound is administered after administration of the anti-VEGF polypeptide antibody.

8. The method of claim 1, wherein the alkylating agent is administered after administration of the taxane compound.

9. The method of claim 1, wherein said taxane compound is paclitaxel.

10. The method of claim 1, wherein said alkylating agent is a platinum compound.

11. The method of claim 10, wherein said platinum compound is carboplatin.

12. The method of claim 1, wherein said anti-VEGF polypeptide antibody is a humanized antibody.

13. The method of claim 1, wherein said anti-VEGF polypeptide antibody is bevacizumab.

14. The method of claim 1, wherein said taxane compound, said alkylating agent, and said anti-VEGF polypeptide antibody are administered by injection.

15. The method of claim 1, wherein said taxane compound is administered more frequently than said anti-VEGF polypeptide antibody, and wherein said anti-VEGF polypeptide antibody is administered more frequently than said alkylating agent.

16. The method of claim 1, wherein each of said taxane compound, said alkylating agent, and said anti-VEGF polypeptide antibody is administered within a 60 day time period.

17. The method of claim 1, wherein said progression-free survival is increased by 25 percent.

18. The method of claim 1, wherein said progression-free survival is increased by 50 percent.

19. The method of claim 1, wherein said progression-free survival is increased by 75 percent.

20. The method of claim 1, wherein said progression-free survival is increased by 100 percent.

21. The method of claim 1, wherein said taxane compound, said alkylating agent, and said anti-VEGF polypeptide antibody are administered under conditions wherein the time to progression is increased.

22. A composition comprising a taxane compound, an alkylating agent, and an anti-VEGF polypeptide antibody.
